(19) United States
(12) Patent Application Publication
(10) Pub. No.: US 2009/0247987 A1
(43) Pub. Date: Oct. 1, 2009

(54) Drainage Catheter

(76) Inventors: Raymond Chevalier, JR., Bloomington, IN (US); Daniel Miller, St. Louis, MO (US); Steve Pickett, Spencer, IN (US); Carla Wiese, Cincinnati, OH (US); Leslie P. Sherwood, Elletsville, IN (US)

Correspondence Address: FAY KAPLUN & MARCIN, LLP, 150 BROADWAY, SUITE 702, NEW YORK, NY, 10038, US

(21) Appl. No.: 12/323,695

(22) Filed: Nov. 26, 2008

Related U.S. Application Data
(60) Provisional application No. 60/991,097, filed on Nov. 29, 2007.

(51) Int. Cl. A61M 25/00

(52) U.S. Cl. 604/525

(57) ABSTRACT

A catheter includes a shaft formed of a first material, the shaft including a shaft lumen extending therethrough from a proximal opening to a distal end of the shaft and an abutting member coupled to the distal end of the shaft, the abutting member being formed of a second material stiffer than the first material and forming an abutting surface at least partially covering a distal end of the shaft lumen in combination with a distal tip coupled to the abutting member, the distal tip including a tip lumen extending therethrough to a distal opening.

### PRIORITY CLAIM

This application claims the priority to the U.S. Provisional Application Ser. No. 60/991,097, entitled “DRAINAGE CATHETER,” filed Nov. 29, 2007. The specification of the above-identified application is incorporated herewith by reference.

### BACKGROUND INFORMATION

Drainage catheters are often inserted percutaneously into abscesses or other structures to drain fluids. The shafts of these catheters are formed in a variety of sizes, durometers, and coatings. Holes located at the distal end of a typical drainage catheter allow fluids surrounding the catheter to enter a lumen through which they flow to the proximal end of the catheter out of the body. To enhance patient comfort, these catheters are generally made of soft biocompatible plastic. The soft material generally results in low column strength, so that a stiffening member is often inserted into a drainage catheter to allow it to be pushed into the body to a desired location. The stiffening member often abuts a surface near the distal end of the lumen at which the lumen tapers or steps down from a larger diameter, proximal portion to a reduced diameter, distal portion.

### SUMMARY OF THE INVENTION

The present invention is directed to a catheter including a shaft formed of a first material, the shaft including a shaft lumen extending therethrough from a proximal opening to a distal end of the shaft and an abutting member coupled to the distal end of the shaft, the abutting member being formed of a second material stiffer than the first material and forming an abutting surface at least partially covering a distal end of the shaft lumen in combination with a distal tip coupled to the abutting member, the distal tip including a tip lumen extending therethrough to a distal opening.

### BRIEF DESCRIPTION OF THE DRAWINGS

FIG. 1 shows a drainage catheter assembly according to the invention;

FIG. 2 shows a cross-sectional view of a catheter according to a first embodiment of the present invention;

FIG. 3 shows a cross-sectional view of a catheter according to a second embodiment of the present invention;

FIG. 4 shows a cross-sectional view of a catheter according to a third embodiment of the present invention;

FIG. 5 shows a cross-sectional view of a catheter according to a fourth embodiment of the present invention; and

FIG. 6 shows a cross-sectional view of a catheter according to a fifth embodiment of the present invention.

FIG. 7 shows a cross-sectional view of a catheter according to a sixth embodiment of the present invention.

FIG. 8 shows a cross-sectional view of a catheter according to a seventh embodiment of the present invention.

### DETAILED DESCRIPTION

The present invention may be further understood with reference to the following description and the appended drawings, wherein like elements are referred to with the same reference numerals. The invention relates to devices for draining fluid from the body. More specifically, the invention relates to a novel construction for a drainage catheter.

Drainage catheters typically are made of either a single material, or with a tip section separate from the catheter shaft. The separate tip section may be made of either a material with a different durometer than the catheter shaft, or a different material altogether.

As described above, stiffening members are often inserted into drainage catheters to aid in insertion. However, as the distal end of such a stiffening member is pressed into the catheter lumen, frictional drag along the outside of the catheter body may result in stretching of the catheter and/or the deformation of the material at the end of the lumen contacted by the stiffening member. In some cases, the stiffening member may become lodged within the tip section, preventing the removal of the stiffening member from the catheter. In such cases, the entire catheter must be removed and replaced, increasing discomfort and the time required for the procedure.

The catheter according to the present invention includes an abutting structure within the distal end of the lumen formed of a material having a higher durometer than that of the material of which the rest of the catheter is formed. The increased durometer of the abutting structure reduces the likelihood of the stiffening member becoming stuck within the tip section. The abutting structure is preferably formed of material of a higher modulus of elasticity than the material of which a shaft of the catheter is formed and may, more preferably be a dissolvable material as described in U.S. Pat. Nos. 5,401,257 and 5,049,138, the entire disclosures of which are hereby expressly incorporated herein by reference.

The abutting structure according to the embodiments of this invention may be fused to the catheter shaft material within the tip section as part of the tipping process by, for example, mechanical bonding (e.g., press fit, or snap fit) and/or chemical bonding (e.g., heat fusion, adhesives, or solvents). Alternatively, the abutting structure may be added to the catheter shaft material in a process other than the tipping process. These alternative processes may include a separate adhesive process, a solvent bonding process, a mechanical fit process, or an over-molding process. An outer diameter of the abutting structure preferably mates with an inner diameter of a portion of the catheter shaft within which it is to reside while an inner diameter of the abutting structure is preferably substantially a desired inner diameter of a portion of the lumen extending through the tip section so that the abutting structure does not interfere with fluid flow through the lumen.

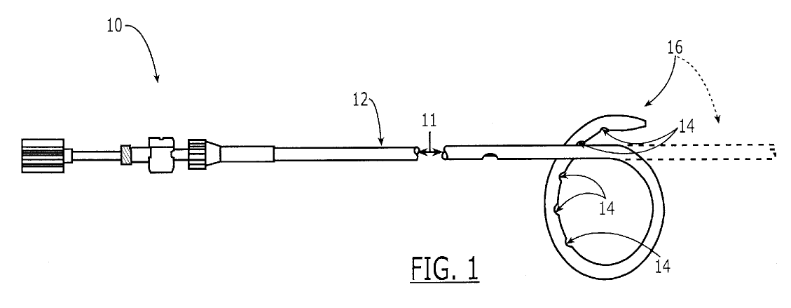

As shown in FIG. 1, a drainage catheter assembly 10 according to the present invention includes a lumen 11 extending through a shaft 12 from the distal end to the proximal end thereof. Generally, multiple holes 14 near the distal end of the catheter shaft 12 allow fluids to drain out of the body. As would be understood by those skilled in the art, in order to assist in maintaining the catheter at a desired location (e.g., within a chamber of a lumen) the tip section 16 of the catheter shaft 12 may have a curled, ‘pigtail’ shape which anchors the drainage catheter assembly 10 at the outlet from the organ chamber to a lumen through which the catheter assembly 10 was introduced thereinto.

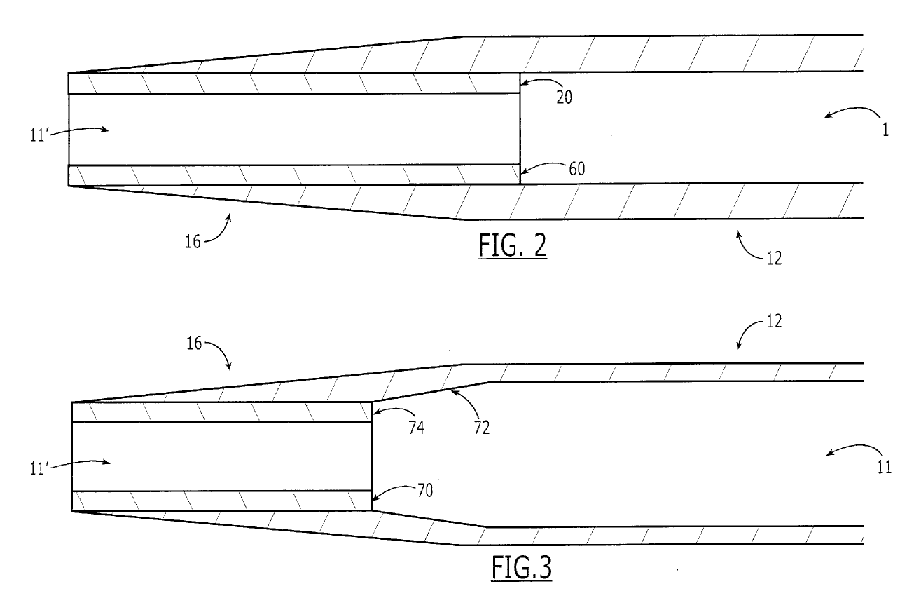

As shown in FIG. 2, a tip 16 including an abutting structure 60 according to a first embodiment of the invention forms a stepped transition 20 in the lumen 11. The abutting structure 60 is formed as a ring mounted within the lumen 11 with the abutting structure 60 being formed of a material stiffer than the material of which the rest of the catheter 10 is formed. An outer diameter of the abutting structure 60 is substantially equal to an inner diameter of the lumen 11 to mate with the inner surface of the catheter shaft 12. The abutting structure 60 extends through the entire tip section 16 to end at the distal opening of the catheter 10. The inner diameter of the abutting structure 60 defines a narrower inner diameter lumen portion 11′ of the lumen 11 extending through the tip section 16. Furthermore, a proximal end of the abutting structure 60 defines an increased stiffness stepped transition 20′ within the lumen portion 11′. Thus, when a stiffening member is pushed against the stepped transition 20, a material of the stepped transition 20 is less likely to deform, thus reducing the likelihood of the stiffening member becoming lodged within the tip section 16, as those skilled in the art will understand. The catheter 10 and the abutting structure 60 may be formed of one of the same material and different materials. The materials for either one may include, for example, any type of thermoplastic polyurethane or low density polyurethane, silicone, ethylene vinyl acetate (EVA), or polyvinyl chloride (PVC), etc. The materials may also include a filler such as, for example, a radiopacifier or anti-microbial agent. The abutting structure may each have a different hardness depending on the material chosen. If a material of the abutting structure 60 is the same as that of the catheter 10, it may only need to be of higher durometer than that of the shaft 12 in order to function as indicated above. Alternatively, the abutting surface may be formed of high density polyethylene (HDPE), nylon, polycarbonate, acrylonitrile butadiene styrene (ABS) and aluminum. Catheters made of thermoplastic polyurethane are commonly found in the durometer range of Shore 72A-Shore 84D, as would be understood by those skilled in the art.

As shown in FIG. 3, a tip 16 including an abutting structure 70 according to a second embodiment of the invention is mounted distally of a tapered tip section 72 of the lumen 11. The abutting structure 70 is formed as a ring of a material stiffer than that of which the rest of the catheter 10 is formed and is mated within the reduced diameter lumen portion 11′. The tapered tip section 72 reduces the diameter of lumen 11 in a distal direction and the abutting structure 70 forms a further stepped reduction in this diameter. The abutting structure 70 according to this embodiment extends through the tip 16 from the distal end of the tapered tip section 72 to the distal end of the catheter 10. As in the previous embodiments, the outer diameter of the abutting structure 70 is substantially equal to the inner diameter of the lumen portion 11′ (i.e., less than this inner diameter of the distal portion of the lumen 11 by only a tolerance amount necessary to facilitate insertion therein). The portion of the lumen 11 extending through the abutting structure 70 may have a substantially constant diameter along its length. Alternatively, the lumen portion 11′ extending through the abutting structure 70 may comprise a tapered diameter. In another alternate embodiment, the tapered tip section 72 may extend along all or a part of the length of the abutting structure 70 with a corresponding taper on an outer surface of the abutting structure 70. As described above, the abutting structure 70 increases the stiffness of the tapered tip section 72 to reduce the likelihood that of a stiffening member being lodged within the tip section 16 or plastically deforming the tip section 16.

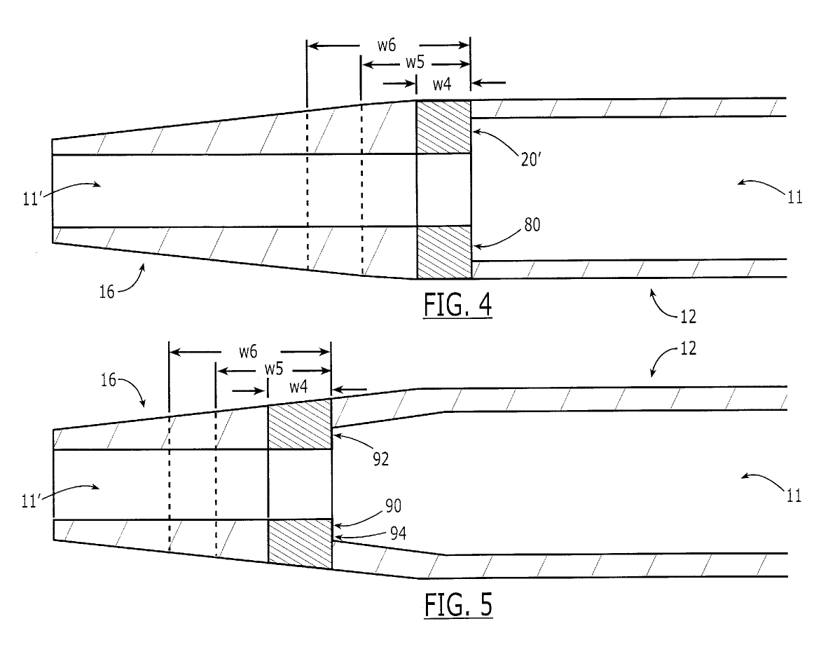

As shown in FIG. 4, an abutting structure 80 according to a third embodiment of the invention is formed at a proximal end of the tip 16. The abutting structure 80 is formed as a ring of higher stiffness material at a point where the catheter shaft 12 is joined to the tip section 16. The abutting structure 80 may be formed with any desired width w (e.g., widths w4, w5, or w6), depending on the requirement of a procedure to be performed therewith. The outer diameter of the proximal end of the abutting structure 80 matches the outer diameter of the distal end of the catheter shaft 12 to form a continuous, smooth transition from the catheter shaft 12 to the tip 16. Similarly, the outer diameter of the distal end of the abutting structure 80 matches the outer diameter of the proximal end of the tip section 16 to also form a smooth transition therewith. The inner diameter of the abutting structure 80 is approximately equal to an inner diameter of the lumen portion 11′ extending through the tip section 16. In this manner, the proximal end of the abutting surface 80 forms an increased stiffness stepped transition 20′ to the lumen portion 11′. Thus, as a stiffening member pushes on the stepped transition 20′ during catheter insertion, the abutting surface 80 reduces the likelihood that the stiffening member will become lodged within the tip section 16.

As shown in FIG. 5, an abutting structure 90 according to a fourth embodiment of the invention is formed at a proximal end of the tip 16. The abutting structure 90 is formed of a relatively stiff material in the shape of a ring joining a tapered transition 30 of the catheter shaft 12 to the tip section 16. Similar to the embodiment of FIG. 4 described above, the abutting structure 90 may have any desired width w (e.g., widths w4, w5, w6, etc.) depending on the desired characteristics of the tip 16. The outer diameter of a proximal end of the abutting structure 90 is substantially equal to that of the distal end of the tapered transition 30 of the catheter body 12 to form a smooth transition therebetween. However, those skilled in the art will understand that any profile of each of the distal end of the catheter body 12, the abutting structure 90 and the tip 16 may be selected to achieve a desired overall contour of the distal end of the catheter 10 so long as transitions between these parts are smooth. The inner diameter of the abutting structure 90 is approximately equal to an inner diameter of the lumen portion 11′ extending through the tip section 16. Thus, a proximal face 92 of the abutting structure 90 forms a stepped transition 94 at an interface with the shaft 12. As in the preceding embodiments, since the abutting structure 90 is formed of a stiffer material than the rest of the catheter 10, the stiffness of the stepped transition 94 is also increased. Thus, as a stiffening member pushes on the stepped transition 94 during catheter insertion, the proximal face 92 of the abutting structure 90 reduces the likelihood that the stiffening member will become lodged within the tip section 16.

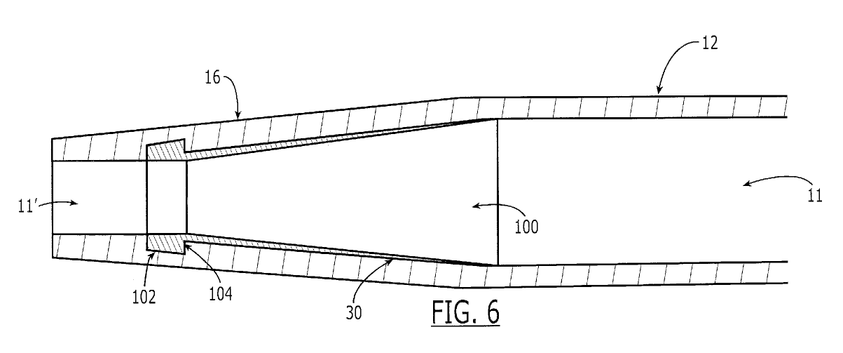

As shown in FIG. 6, an abutting structure 100 according to a fifth embodiment of the invention is formed as a liner of shell within the lumen portion 11′ of the tip 16. The abutting structure 100 is formed of a material of increased stiffness relative to the material of which the catheter 10 is formed and extends along a tapered transition 30 of the tip 16. An outer diameter of the abutting structure 100 mates with an inner diameter of the tapered transition 30 with a tapered proximal thickness to create a smooth inner surface transition from the lumen 11 to the lumen portion 11′, as those skilled in the art will understand. The abutting structure 100 further comprises a locating feature 102 which locks in a mating recess 104 formed within an inner diameter of the tip section 16 adjacent a distal end of the tapered transition 30. Thus, when the locating feature 102 is seated within the mating recess 104, the abutting structure 100 assumes a locked configuration relative to the tip section 16, thereby increasing the stiffness of the tapered transition 30 and forming a hard shell around the tapered portion of the lumen portion 11′.

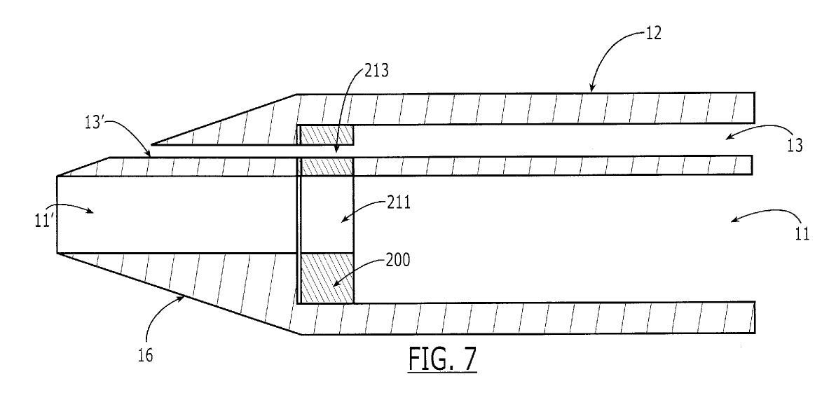

FIG. 7 shows an abutting structure 200 according to a sixth embodiment of the present invention formed at a proximal end of the tip 16 of a shaft 12 of a multilumen catheter. The abutting structure 200 is also formed as a ring of a higher stiffness material relative to the catheter shaft 12 and is situated adjacent a joint between the catheter shaft 12 and the tip 16. Specifically, the abutting structure 200 extends across the distal end of the lumen 11, past a partition dividing the lumen 11 from a second lumen 13 and abuts a portion of the wall of the lumen 13 formed by an outer wall of the shaft 12. In this embodiment, the abutting structure 200 is located proximally of the tapered tip section 16. The abutting structure 200 further comprises a first lumen 211 extending therethrough and substantially aligning with the lumen 11 so that, when the abutting structure is positioned as noted above, the lumen 211 connects the lumen 11 to the lumen 11′. A lumen 213 extending through the abutting structure 200 further aligns with the lumen 13, connecting the lumen 13 to a lumen 13′ extending through the tip 16. Those skilled in the art will understand that the lumens 13, 213 and 13′ open to the distal end of the tip 16 and may be used, for example, to provide aspiration, supply fluids, etc. The lumens 11, 211 and 11′ may then be used for drainage as described above.

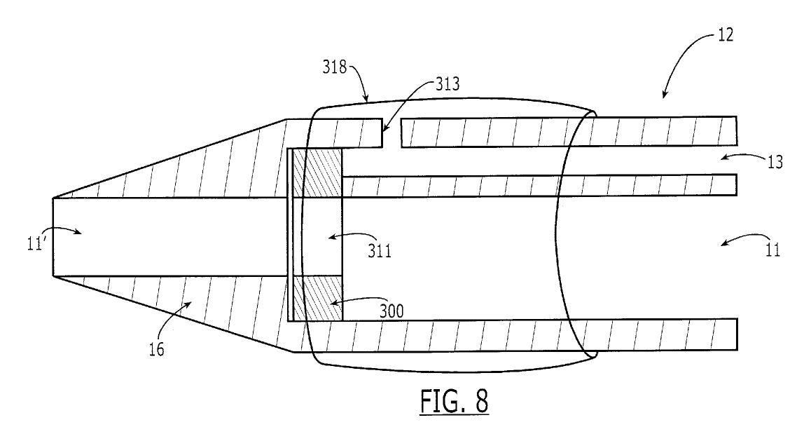

FIG. 8 shows an abutting structure 300 according to a seventh embodiment of the present invention wherein the abutting structure 300 is formed as a ring at a distal end of the catheter shaft 12. Similar to the previously described embodiments, the abutting structure 300 is formed as a ring of a higher stiffness material located between the catheter shaft 12 and the tip 16. The abutting structure 300 extends across the distal end of the inner diameter of the lumen 11, through a partition dividing the lumen 11 from the lumen 13 to abut a portion of the wall of the lumen 13 formed by an outer wall of the shaft 12. In this embodiment, the abutting structure 300 is located proximally of the tapered tip section 16. The abutting structure 300 comprises a lumen 311 extending therethrough substantially aligning with the lumen 11 to connect the lumen 11 to the lumen 11′ of the tip 16, thus providing a stepped diameter from the lumen 11 to the lumen 11′. A portion of the abutting structure 300 extends across and closes a distal end of the lumen 13. The catheter shaft 12 further comprises an inflation port 313 connecting the lumen 13 to an exterior of the shaft 12 which is enveloped in a balloon 318. Thus, the provision of inflation fluid to the lumen 13 inflates the balloon 318 to, for example abut adjacent tissue and lock the catheter in position.

The abutting structures of the catheters according to the embodiments of the present invention are shown in the appended figures as rings which substantially match the internal shape of the lumen of the catheter. However, alternative designs other than rings (e.g., projections into the lumen) may also be used so long as they provide a stiffer surface for engaging a stiffening member inserted into the catheter. These shapes preferably conform to the shape of the lumen of a single lumen catheter. However, those skilled in the art will understand that for multiple lumen catheters, the shape of an abutting surface may conform to a shape of an outer wall of the catheter, to the shapes of the lumens themselves or may simply form projections into the lumens against which the stiffening member(s) will push. Additionally, the abutting structures may be a variety of sizes (e.g., widths, thicknesses, or diameters) for use in a variety of catheters, for extra stiffening capacity, or for ease of manufacture. Further, one or more abutting structures may be added to multi-lumen drainage catheters as well.

The concept of adding a stiffer material to a catheter shaft may also be used for other products (e.g., venous access catheters such as dialysis, tunneled central, and PICC (peripherally inserted central catheters)) that have flexible shafts into which an insertion tool or other device is inserted to impart a force to the product through contact with an internal structure which might be damaged if not formed of a material more stiff than that of which the rest of the catheter is formed. For example, such a construction may be employed for pacemaker leads, aneurism coils, removable core guidewires, introducer sheaths for vascular access, etc. Further, the secondary material may be added along the entire length of the shaft or only along any portion of the shaft as desired, such as the tip section as shown in the exemplary embodiments.

The present invention has been described with reference to specific embodiments, and more specifically to an abutting structure for use with drainage catheters. However, other embodiments may be devised that are applicable to other medical devices and procedures, without departing from the scope of the invention. Accordingly, various modifications and changes may be made to the embodiments, without departing from the broadest spirit and scope of the present invention as set forth in the claims that follow. The specification and drawings are accordingly to be regarded in an illustrative rather than restrictive sense.

What is claimed is:

1. A catheter including:
a shaft formed of a first material, the shaft including a shaft lumen extending therethrough from a proximal opening to a distal end of the shaft;
an abutting member coupled to the distal end of the shaft, the abutting member being formed of a second material stiffer than the first material and forming an abutting surface at least partially covering a distal end of the shaft lumen; and
a distal tip coupled to the abutting member, the distal tip including a tip lumen extending therethrough to a distal opening.

2. The catheter of claim 1, wherein an inner diameter of the abutting member lumen is smaller than an inner diameter of the shaft lumen.

3. The catheter of claim 1, wherein the distal tip is formed of the first material.

4. The catheter of claim 1, wherein the distal tip is formed of a third material, the first material being stiffer than the third material.

5. The catheter of claim 1, wherein an outer surface of the shaft member and an outer surface of the abutting member are matched to form a smooth exterior of the catheter.

6. The catheter of claim 5, wherein the outer surface of the shaft is substantially cylindrical and the outer surface of the abutting member is substantially cylindrical with outer diameters of the shaft and the abutting member being substantially equal.

7. The catheter of claim 5, wherein the outer surface of the shaft is substantially cylindrical and the outer surface of the abutting member is substantially conical with an outer diameter of the shaft and an outer diameter of a proximal end of the abutting member being substantially equal, the outer diameter of the abutting member tapering from a maximum at the proximal end to a minimum at a distal end thereof, with an outer diameter of the distal end of the abutting member being substantially equal to an outer diameter of a proximal end of the tip.

8. The catheter of claim 1, wherein an inner diameter of at least a portion of the abutting member lumen tapers to a minimum diameter at a distal end of the abutting member.

9. The catheter of claim 1, wherein the shaft lumen includes a tapered transition immediately proximal to the abutting member, the inner diameter of a portion of the shaft lumen remaining substantially constant from the proximal opening to a proximal end of the tapered transition and gradually decreasing through the tapered transition.

10. The catheter of claim 9, wherein an inner diameter of the abutting member lumen is substantially constant along its length.

11. A catheter including:
a shaft formed of a first material, the shaft including an integrally formed distal tip and a shaft lumen extending therethrough from a proximal opening to a distal opening in the distal tip; and
an abutting member coupled within a distal portion of the shaft lumen and extending through the distal portion to the distal opening in the distal tip, the abutting member being formed of a second material stiffer than the first material and extending into the shaft lumen from an inner surface of the shaft to form at least one abutting surface within the shaft lumen.

12. The catheter of claim 11, wherein the shaft lumen and the abutting member lumen are substantially cylindrical, an outer diameter of the abutting member being substantially equal to an inner diameter of the shaft lumen.

13. The catheter of claim 11, wherein the shaft lumen includes a tapered transition immediately proximal to the abutting member, the inner diameter of a portion of the shaft lumen remaining substantially constant from the proximal opening to a proximal end of the tapered transition and gradually decreasing through the tapered transition.

14. The catheter of claim 13, wherein the inner diameter of a portion of the shaft lumen extending distally from a distal end of the tapered transition to the distal opening remains substantially constant.

15. The catheter of claim 14, wherein the abutting member lumen is substantially cylindrical and an outer diameter of the abutting member is substantially equal to an inner diameter of the portion of the shaft lumen extending distally from the distal end of the tapered transition.

16. The catheter of claim 14, wherein a length of the abutting member is substantially equal to a length of the portion of the shaft lumen extending distally from the distal end of the tapered transition.

17. The catheter of claim 15, wherein an outer diameter of the tip tapers to a minimum diameter at the distal opening and wherein a thickness of the wall of the shaft decreases as the outer diameter of the tip decreases.
